(19) United States
(12) Patent Application Publication
(10) Pub. No.: US 2018/0361168 A1
(43) Pub. Date: Dec. 20, 2018

(54) Electronic Circuit for Magnetic Neurostimulation and Associated Control

(71) Applicant: Stefan M. Goetz, Durham, NC (US)

(72) Inventor: Stefan M. Goetz, Durham, NC (US)

(21) Appl. No.: 16/013,278

(22) Filed: Jun. 20, 2018

(30) Foreign Application Priority Data
Jun. 20, 2017 (DE) 102017113581.2

(51) Int. Cl. A61N 2/02; A61N 2/00

(52) U.S. Cl. CPC A61N 2/02; A61N 2/006

(57) ABSTRACT

One method employs an electronic circuit with at least two electrical switches and at least one electrical energy store, wherein at least one control unit transmits electrical signals for controlling the at least two electrical switches, directed as coded electrical signals to at least one decoder, and decoded by the at least one decoder into a respective switch state to be set of an individual one of the switch control signals describing at least two switches, wherein the respective switch control signals are directed to the respective at least two switches and there correspondingly converted, wherein at one output of the electrical switch for excitation of at least one stimulation coil current pulses with a total length of less than 5 ms are provided, so that the at least one stimulation coil generates magnetic field pulses with a magnetic flow density of 0.1 to 10 Tesla.

### CROSS-REFERENCE TO RELATED APPLICATIONS

This application claims priority to German Patent Application No. 10 2017 113 581, filed Jun. 20, 2017, entitled “Elektronische Schaltung zur magnetischen Neurostimulation and zugehorige Steuerung,” the entirety of which is hereby incorporated by reference in its entirety.

### FIELD

The invention relates to generation of stimulation pulses for inductive neurostimulation, in particular circuits for generating magnetic stimulation pulses and their control, including for peripheral and transcranial magnetic stimulation (TMS).

### BACKGROUND

Magnetic stimulation, in which particular cells are stimulated by externally applied electromagnetic fields in body tissue, including first of all neurons, for example in nerves and muscle cells, is considered the only current pain-free non-invasive method for stimulating neurons in the brain of a patient. In addition, in recent years it has also been increasingly used in the periphery of the nervous system, for example for medical rehabilitation, diagnosis, nervous system research, and the frequently transsynaptic interaction of peripheral and central nervous signals.

Magnetic stimulation is normally based on the principle of magnetic induction. A mobile or permanently installed conductor coil, the so-called stimulation coil, is placed near a subject, patient, or an animal to be stimulated and a time-variable current flows through it so that a correspondingly time-variable magnetic field is established, which penetrates the tissue to be stimulated and induces electric fields in it. These fields, and the currents caused by them, in turn stimulate neurons, muscle cells and other stimulable structures. The prior art is stimulation coils that satisfy particular stimulation properties especially well, such as penetration depth, a particularly high focality of stimulation by spatial concentration of the induced fields, or specific stimulation of multiple targets or areas [Z.-D. Deng, S. H. Lisanby, A. V. Peterchev (2013). Electric field depth-focality tradeoff in transcranial magnetic stimulation: Simulation comparison of 50 coil designs. Brain Stimulation, 6(1):1-13.]. An advantage of inductive magnetic stimulation is the lack of contact, since fields can also be induced in the target tissue over a certain spatial distance. In addition, in contrast to electrical stimulation through electrodes the method is almost completely pain-free, since high current densities in areas with high nociceptor density, such as the skin, are avoided. For these reasons the method is also well-suited for stimulation of deep tissue structures, such as the cerebral cortex through the cranial bone, and for pain-free muscle stimulation.

Besides introducing individual signals in the neurons, muscle cells or other stimulable structures in the body, magnetic stimulation also allows so-called neuromodulation. With the aid of certain so-called protocols, usually particular pulse rhythms, for example, the stimulability of neuronal networks can be specifically changed. Neuromodulation is currently one of the leading uses of magnetic stimulation in the medical field and in science. However, the neuromodulatory effect sizes achievable with conventional devices are very low. The low effect sizes of neuromodulation achievable so far with magnetic pulses is a central problem of the medical use of magnetic stimulation.

Although the potential fluctuations in the neuron membrane necessary for the stimulation are in the range of only a few millivolts, currently available stimulation devices nonetheless require pulse powers in the megawatt range with a waste heat of sometimes several kilowatts. The stimulation coil usually warms up in the pulse operation so much that the length of use is frequently limited to just a few minutes.

One very effective way for increasing energy efficiency and reducing heat development has been discussed in the scientific literature [S. M. Goetz, N. C. Truong, M. G. Gerhofer, A. V. Peterchev, H.-G. Herzog, T. Weyh (2012). Optimization of magnetic neurostimulation waveforms for minimum power loss, Proc. IEEE EMBC 2012:4652-4656; S. M. Goetz, C. N. Truong, M. Gerhofer, A. V. Peterchev, H.-G. Herzog, T. Weyh (2013). Analysis and Optimization of Pulse Dynamics for Magnetic Stimulation. PLOS ONE 8(3):e55771.]. In contrast to sinusoidal current courses of the pulse in the stimulation coil, so-called pulse forms, new, correspondingly optimized current courses for inducing the stimulating fields are proposed.

Much stronger neuromodulating pulse forms have also been determined and experimentally confirmed [S. M. Goetz, B. Luber, S. H. Lisanby, C. I. Kozyrkov, W. M. Grill, and A. V. Peterchev (2013). Enhancement of rTMS neuromodulatory effects with novel waveforms demonstrated via controllable pulse parameter TMS (cTMS). 52nd Meeting of the American College of Neuropsychopharmacology, Hollywood, Fla.; J. Taylor, C. K. Loo (2007). Stimulus waveform influences the efficacy of repetitive transcranial magnetic stimulation. Journal of Affective Disorders, 97(1-3):271-276. S. M. Goetz, B. Luber, S. H. Lisanby, D. L. K. Murphy, I. C. Kozyrkov, W. M. Grill, A. V. Peterchev (2016). Enhancement of Neuromodulation with Novel Pulse Shapes Generated by Controllable Pulse Parameter Transcranial Magnetic Stimulation. Brain Stimulation, 9(1):39-47].

While the pulse form has been identified as a key parameter for various applications of magnetic stimulation, for basic reasons devices available from the prior art are not able to change the pulse form. The main reason for the great limitation of the pulse form, usually to sinusoidal courses, is the high currents and the high spectral shares of stimulable pulses. A conventional circuit topology for generating controlled magnetic pulses of high intensity for transcranial magnetic stimulation is shown in FIG. 1. It comprises an oscillation circuit from a high-voltage capacitor C, such as a film capacitor, and a stimulation coil L, connected through a switch Q, such as a transistor. A charging circuit charges the capacitor C to a voltage of several 1000 V. The energy content of the capacitor can then be a few 100 J. A closing of the switch Q then initiates the power flow through the coil L and there generates the stimulation field. However, most of the energy is lost as waste heat through the resistor R.

A further development that enables high flexibility is the circuit topology shown in FIG. 2 [A. V. Peterchev, D. L. K. Murphy, S. H. Lisanby (2011). Repetitive Transcranial Magnetic Stimulator with Controllable Pulse Parameters. Journal of Neural Engineering, 8(3):036016]. In this half-bridge configuration, the stimulation coil L is connected through switches Q₁ and Q₂ alternatingly with the high voltage capacitors Cₚ and Cₘ. The energy taken from the first capacitor Cₚ with the closing of the switch Q₁, which is not converted to the magnetic pulse in the coil L, can be at least partly fed back to the second capacitor Cₘ. With the subsequent closing of the switch Q₂, the capacitor Cₘ then conversely feeds the capacitor Cₚ, so that the waste heat losses are less compared with the circuit configuration of FIG. 1. Additionally, the use of two independent capacitors Cₚ and Cₘ each with separate charging circuit raises the flexibility in generating the pulse forms.

Further developments of this technology are explained in the scientific literature [A. V. Peterchev (2011). Circuit Topology Comparison and Design Analysis for Controllable Pulse Parameter Transcranial Magnetic Stimulators. Proc IEEE NES, 5:646-649; A. V. Peterchev, K. D'Ostilio, J. C. Rothwell, D. L. K. Murphy (2014). Controllable pulse parameter transcranial magnetic stimulator with enhanced circuit topology and pulse shaping. Journal of Neural Engineering, 11(5):056023.] and the patent literature [U.S. Pat. No. 7,753,836; U.S. Pat. No. 7,946,973]. These technologies use coupled switchable oscillators to provide the high electrical power for a pulse and raise the flexibility to section-wise rectangular pulses with more than two phases, but they are not able to generate arbitrary pulse courses similar to a digital/analog converter. However, such flexibility proves to be difficult to enable the pulse forms recognized as particularly advantageous, e.g., for high efficiency and lower coil heating, high neuromodulation strength, or low acoustic emissions.

For this reason, corresponding technologies able to generate arbitrary pulse forms are the subject of keen research and development activity. In EP 0 958 844, Schweighofer et al. describe a technology with the aid of which a semiconductor circuit and pulse width modulation should generate random pulse shapes for stimulation of neurons. Insulated gate bipolar transistors (IGBT) can be used as semiconductors. It is true that contrary to most other established high-voltage switches these semiconductor switches can also be switched off, but they have the problem of a very low switch speed. The possible switch speed for the pulse width modulation is thus in about the same frequency range as the basic frequency of typical TMS pulses between 3 kHz and 8 kHz. However, for a halfway precise generation of a pulse the switch rate of the semiconductor should be at least one magnitude above the highest frequency component of the pulse form. As it subsequently turned out, at such high switch rates IGBT are loaded far beyond their specifications, the switch losses rise disproportionally, and the IGBTs rapidly wear out or suffer permanent damage already after a few pulses. For this reason, not a single device of this technology that functions over a long period is known.

FIG. 3 presents an alternative technology that to generate the high pulse voltage of several thousand volts and high necessary switch rate of at least several hundred kilohertz presents the pulse voltage as the sum of the output voltage of a plurality of bridge modules (FIG. 4) [S. M. Goetz et al. (2012). Circuit topology and control principle for a first magnetic stimulator with fully controllable waveform. Proc. EMBS. 4700-4703; U.S. Ser. No. 13/030,239]. Each bridge module, or module for short, consists of multiple semiconductor switches, hereafter switches for short, and at least one electrical energy storage element, energy store for short, such as a capacitor. The switches make it possible to electrically connect in series a particular number of electrical stores relative to the output, where the stimulation coil is connected electrically conducting, and electrically decouple the other electrical stores at least with one of their respective connections, so that these neither accept nor give up a charge relative to the stimulation coil. In this way, the voltage at the stimulation coil can be changed very precisely in stages and through switch modulation intermediate values of the voltage can also be created with an accuracy below one step level. Each module can therefore assume multiple states determined by the states of the individual switches of the module (e.g., conducting or non-conducting). The states of the switches of all modules define the overall state of the system.

Since this technology can increase or reduce the output voltage in very small stages and can also alternatingly switch the individual semiconductor switches so that the switch load can be distributed through all semiconductors, in principle it can itself generate pulses with very high frequency components with low distortion and avoids the key problems of high-voltage circuits such as those from Schweighofer et al. In addition, due to the higher availability using a large number of inexpensive low-voltage semiconductors is much more favorable than a few high-voltage semiconductors.

The technology named above allows only a series connection of modules, which is why each module is designed to the maximum current to be expected. However, due to the almost exclusively inductive load in the form of the stimulation coils, since the greatest current usually flows mainly at low voltages at this time the current is provided only by a small number of modules and their module capacitors. The internal resistance and available capacity are therefore very unfavorable. A further development of the above technology enables a dynamic switch between series and parallel connection of the modules, so that a majority of the modules can be switched in parallel at low voltages while at higher voltages more and more modules switch to a series connection [U.S. Ser. No. 13/990,463].

Switching state of a module (state for short) identifies the way in which the module's switch or switches are activated or deactivated to connect or explicitly not connect at least one electrical energy store of the module electrically conducting with at least one electrical energy store of at least one other module of a different type, the so-called connectivity (i.e., open circuit and/or separate connection), so that several modules jointly generate electrical voltage. Examples of possible connectivities of electrical energy stores are parallel connection and series connection, combinations of electrical energy stores, and energy stores unconnected or connected with only one contact. Modules are usually able to present at least two of the following states or connectivity forms through electrical switches of the modules:

(a) The at least one electrical energy store of a module is switched in series with the aid of electrical switches with the at least one energy store of another module;

(b) the at least one electrical energy store of a module is switched in parallel with the aid of electrical switches with the at least one energy store of another module;

(c) the at least one electrical energy store of a module is bypassed with the aid of electrical switches, which means that the at least one electrical energy store of a module is connected electrically conducting only with a maximum of one of its at least two electrical contacts with an electrical energy store of another module, so that no closed circuit with an electrical energy store of another module is present.

Although these technologies in principle enable generation of arbitrary pulse forms and, in contrast to alternative approaches, do not unduly burden any power semiconductors or yield low pulse quality, they have critical drawbacks. A large number of bridge modules are needed for generating typical magnetic stimulation pulses with a voltage of several thousand volts and currents of several thousand amperes. Each of these modules uses multiple individual switches, usually between four and eight switches, which should be controllable independent of each other to generate free pulse forms. The high number of switches to be independently controlled leads to a high number of control signals to be provided. Typical microprocessors, microcontrollers, signal processors and similar components usually provide some 30 to 80 inputs and outputs. Higher numbers of signals are used in a few other technical applications. In addition, current housing types limit the number of inputs and outputs that can be directed outward from a semiconductor chip.

For a structure of, for example, only 40 modules with voltage of 75 V each, which can generate an output voltage of 3000 V, the number of control lines necessary from the control unit implemented in one or more microprocessors adds to 160 to 320, which furthermore are needed with high time accuracy and high data rate. Typical update rates of the control lines are less than 1 ms, preferably below 1 μs, to guarantee low distortions of the pulse form. Due to the high switching speed of the field effect transistors generally used of normally below 100 ns, a time accuracy of the signals well below this switching speed is also necessary. Consequently, a very accurate signal generation of a large number of parallel channels with high signal rate in real time is necessary. Technically, this is only feasible with very expensive and rare components. The synchronization of multiple processors typical in other technical fields to generate the large number of parallel channels is only possible with great difficulty due to the necessary high time accuracy, which typically prohibits jitter above 100 ns.

A second central problem of this flexible technology from the prior art is the high susceptibility to electromagnetic disturbances. The current strengths controlled by the semiconductor switches usually exceed several thousand amperes and are led to and away in direct proximity to the control signals. For this reason, the flexible TMS technology named above has the intolerable problem that the current to be controlled reacts upon the control line and the signals generated by it in the control lines are even magnitudes higher than the desired control signals of the device's control unit. However, for many areas of use a stimulator that is no longer fully controlled by the control unit according to the specifications but instead itself exerts an influence is impractical for many areas of use, but due to the high energies and work on the nervous system, for example the brain, this is safety-critical.

Comparable semiconductor circuits from power engineering do control equally high powers in the megawatt range and above, but the semiconductor switches from the flexible TMS technology named above, in contrast to similar technologies from power engineering, almost without exception use extremely slow high-voltage components like insulated gate bipolar transistors (IGBT) and thyristors, very short response times, and small gate capacities. The high-voltage components in power engineering need even at the control input, the gate, currents of several amperes for the switch between the conductive and blocking state, due to which the ratio of controlled current and current to be controlled during the switching is relatively low. Since electromagnetic disruptions consequently must likewise be in the ampere range already to have a noticeable influence, the gate of the semiconductors of similar power engineering switches is much less sensitive to self-generated electromagnetic disruptions. Besides this insensitivity due to the high necessary power currents, the corresponding semiconductors are slower by several orders of magnitude. Consequently, highly frequent or short electromagnetic interferences (often called salt-and-pepper noise or interference peaks) have a negligible effect since the inertia of the components could not follow this.

The use of only one signal channel per half bridge of two switches electrically connected in series, as is customary in power engineering, would indeed reduce the number of necessary lines but is usually not usable for the flexible TMS technologies discussed above, because with this one binary signal channel the two switches of the bridge are activated in alternation so that one switch of the bridge is always conductive. However, for many pulses in magnetic stimulation, given unknown or not exactly known electrical properties of the stimulation coil the state is also needed in which neither of the two switches is activated by the control unit but instead only diodes (free-wheeling diodes, see U.S. Ser. No. 13/990,463; U.S. Ser. No. 13/030,239; S. M. Goetz et al. (2012). Circuit topology and control principle for a first magnetic stimulator with fully controllable waveform. Proc. EMBS. 4700-4703) as rectifier elements direct the current according to its direction of flow. This state is usually called passive, since no deliberate activation of switches by the control unit is needed and the control unit does not have to know the current at the stimulation coil to determine the commutation time. Instead, the passive state can be used for discharging the energy of the inductivity of the stimulation coil through specific deactivation of switches.

Since the semiconductors of the flexible TMS technologies named above, by contrast, are usually implemented through very sensitive low-voltage semiconductors, in particular field effect transistors (FET), which need response times in the nanosecond range and very low control currents, typical solutions from power engineering are only usable to a very limited extent.

Optical control lines instead of electrical ones, as are widespread in power engineering, would greatly raise the cost of the stimulation device and make it unaffordable for customary doctors' offices or scientific laboratories. In addition, in contrast to power engineering directly optically controlled semiconductors are not widespread for the low-voltage range.

The object of the present invention is therefore to address at least some of the disadvantages of the prior art. At a minimum, at least an alternative to known solutions should be suggested.

### SUMMARY

This object is achieved with a method and device with the features of the respective independent claims. Further embodiments emerge from the dependent claims and the description.

A method is proposed for generating short current pulses by means of an electronic circuit with at least two electric switches and at least one electric energy store, where at least one electronic control unit transmits electrical signals to control the at least two electric switches that are coded on the basis of a predetermined pattern for switch states to be set of the at least two electrical switches, sent as coded electrical signals over an electrical signal transmission line to at least one decoder, and decoded by the at least one decoder into a switch state to be set of an individual one of the switch control signals describing at least two switches, where the respective switch control signals are directed to the respective at least two switches and there correspondingly converted, wherein at one output of the electrical switch for stimulation of at least one stimulation coil current pulses with a total length of less than 5 ms are provided, so that the at least one stimulation coil generates magnetic field pulses with a magnetic flow density of 0.1 to 10 Tesla, which create electrical currents in body tissue according to the principle of electromagnetic induction that through stimulation trigger at least one action potential of nerve and/or muscle cells, where the at least one stimulation coil is designed such that a magnetic field generated by it can penetrate the body tissue.

The at least one electrical energy store is designed to store part of or the entire energy needed for the magnetic field pulses.

The electrical stimulation currents caused by the magnetic field of the stimulation coil are at least a tenth of and a maximum of ten times the stimulation currents needed for a stimulation of the cells.

In a preferred embodiment, an average data rate or an average redundancy of the coded electrical signals is lower than a corresponding average data rate or an average redundancy of the signals decoded by the at least one decoder to switch control signals.

In addition, a device is provided for generating short current pulses by means of an electronic circuit with at least two electrical switches and with at least one electrical power supply, where the device at a minimum comprises: an electronic control unit configured to transmit electrical signals to control the at least two electrical switches, to be directed as coded electrical signals over an electrical signal transmission line to at least one decoder, the at least one decoder, configured to decode the coded electrical signals into a respective switch state to be set of an individual one of the switch control signals describing at least two switches, where the respective switch control signals with conversion at the respective at least two switches are such that at one output of the electronic circuit for generation of at least one of the stimulation coils current pulses with a total length of less than 5 ms are provided so that the at least one stimulation coil given excitation with these current pulses generates magnetic field pulses with a magnetic flow density of 0.1 to 10 Tesla, which cause electrical currents in body tissue according to the principle of electromagnetic induction, which through stimulation trigger at least one action potential of nerve and/or muscle cells.

In a possible embodiment, the device according to the invention comprises at least one coding unit, configured to code electrical signals sent or to be sent by the electronic control unit on the basis of a predetermined pattern for switch states to be set of the at least two electrical switches and directed as coded electrical signals over an electrical signal transmission line to at least one decoder.

In a possible embodiment, the at least one coding unit is integrated into the control unit or the at least one coding unit is part of the control unit. In another embodiment, the at least one coding unit is integrated into at least one electronic circuit subordinate to the at least one electronic control unit. Alternatively, the coding unit can also be provided as a separate unit, for example in the form of an encoder, or comprise an encoder.

According to one embodiment of the device according to the invention, the average data rate or the average redundancy of the electrical signals is lower than the corresponding average data rate or average redundancy of the signals decoded to switch control signals by the at least one decoder.

The present invention presents a novel solution for overcoming the problems in the prior art. First of all, the invention enables a reduction of the control lines, so that control of the system in the form of at least one control unit with conventional microprocessors from the prior art can occur. In addition, it reduces the susceptibility to electromagnetic cross-feed or interference from both external sources and in particular through its own pulse current generated by the magnetic stimulator and its switch transitions and switching peaks.

Although for generation of random pulse forms each semiconductor switch must be individually controlled, the totality of the control lines is highly pattern-affected. In addition, not all combinations of switch states are necessary for the goal of generating random pulse shapes. While these properties are deliberately used in the present invention, the previous consensus in the technical community of magnetic stimulation was that the high number of control lines represents the necessary price for obtaining the previously unknown flexibility. Usable patterns in the switch states were not considered to be obvious, since the high number of switches and the dependence of the switches' switch states among each other lead to a high complexity that obscures the view of simple structures. For this reason, the control signals in existing systems are generated exclusively computer-aided and not manually, so that even after a deliberate numerical entropy analysis only the Shannon redundancy would be known but no natural compression possibilities would be clear. The complexity due to the high number of signals is further increased by switch modulation, which causes very fast changes between different and usually non-recurring switch states.

For the reduction, at least at one place of the transmission of the control signals the invention uses a coding so that one signal is no longer used per switch to be independently controlled; instead, the control signal represents states and partial states. A purely parallel coding, a purely serial coding, and a mixture of parallel and serial coding can be used for the reduction of redundancy in the sense of the invention.

In a purely parallel coding, the totality of all coded signals at a particular time clearly defines the complete state of all switches. In a purely serial coding only one signal channel is present, the signal of which, as a serial signal, defines the state of all switches not at a particular time but by the sum of signals communicated in succession. The signals communicated in succession determining the state of all switches must be completely communicated at the time the switches must assume the corresponding state. However, the signals communicated in succession defining the state of all switches need not follow each other without a gap; instead, they can be interrupted or interlocked with each other (so-called interleaving), as is used, for example, in the compact disc (CD), e.g., according to the Red Book standard. With a mixture of parallel and serial coding, the state of each individual switch is defined in more than one signal channel and at more than one time. Consequently, s signals communicated in succession on k parallel channels define the state of each switch.

In addition, a string coding can be used that likewise can be present as purely parallel, purely serial, or mixed parallel serial coding. With string coding, for the clear definition of the status of each switch the status of each switch at one or more times in the past is also needed. In the simplest case, string coding is a differential coding in which the signal is presented as the difference from a preceding signal. More complicated string codings are also possible, which can be implemented, for example, with shift registers. Reed Solomon codes or convolution codes are examples of a more complicated string coding.

According to a first aspect of the invention, the coding unit is realized by the at least one control unit or at least an electronic circuit subordinate to it, i.e., the at least one control unit or at least one electronic circuit subordinate to it generates a coding that needs fewer signal lines and/or a lower data rate than the control of each switch with one line each in the prior art. Through the lower number, the control signals in their totality can be generated for the complete system in a control unit and electronically outputted by it despite its generally lower number of inputs and outputs.

One or more electronic circuits are considered as subordinate to one or more control units if these one or more electronic circuits receive and process electrical control signals from the one or the more control units.

In addition, optionally one or more separate encoders can be implemented that perform the coding based on the control signals of the at least one control unit or at least an electronic circuit subordinate to it in the form that its at least one output signal needs a lower data rate than a control of each circuit with one line each according to the prior art.

At least one decoder determines from the at least one encoded signal the necessary state of at least one electrical switch. A decoder in the sense of the invention can receive only a part of the at least one signal; in the case of signals transmitted through parallel channels, for example, only a few channels; in the case of signals transmitted serially, for example, by evaluation of only a few transmit symbols or bits from the total data stream of the at least one channel; in the case of, for example, channel multiplexing according to the code division multiplex access (CDMA) method, only one or a few of the channels can be extracted from the at least one signal. It is also advantageous in the sense of the invention if the at least one decoder is spatially situated near the at least one switch whose state the corresponding decoder determines. This spatial proximity can also be supplemented by suitable circuit layout to reduce electromagnetic interference.

In an embodiment, the device according to the invention comprises at least one decoder per module, whereas a given decoder of a module is designed in each case to receive only a subset of a totality of the coded electrical signals received by the decoders, and/or at least one decoder per intermodule connection, where a respective decoder of an intermodule connection is designed in each case to receive only a subset of the totality of the coded electrical signals received by decoders, and/or at least one decoder per intermodule connection unit, where a given decoder of an intermodule connection unit is designed in each case to receive only a subset of the totality of the coded electrical signals received by decoders.

In a possible embodiment the subsets of the totality of signals received by decoders are not identical.

In another embodiment, the subsets of the decoders of the totality of the signals received by decoders are pairwise disjoint.

In another embodiment, the device according to the invention also comprises at least one channel coder.

The at least one channel coder is designed to obtain electronic signals from the at least one encoder.

The at least one channel coder can be integrated with the at least one electronic control unit or at least one of the at least one electronic circuit subordinate to an electronic control unit.

In a possible embodiment, the electronic circuit has at least two modules, each comprising at least one electrical energy store and at least one electrical switch, where the at least two modules can assume at least two of the following switching states:

the at least one electrical energy store of a module is connected in series with the aid of the electrical switch with the at least one energy store of another module;

the at least one electrical energy store of a module is connected in parallel with the aid of the electrical switch with the at least one energy store of another module;

the at least one electrical energy store of a module is circumvented with the aid of the electrical switch in the form of a bypass, which means that the at least one electrical energy store of a module is only connected electrically conducting with at most half of its at least two electrical contacts with an electrical energy store of another module, and thereby no closed circuit with an electrical energy store of another module is present.

Advantageously, the invention uses a so-called codebook, which assigns a coding or an entry to each needed state of the system or parts of the system, such as modules or intermodule connections (comprising the switches of a module and its direct neighbors, i.e., a module that is electrically interconnected directly with the former that can create the direct electrical connections between the electrical energy stores of the two modules). The inventor realized that in addition states are present in certain systems that are not just used only rarely or not at all, but rather the use of which is even detrimental for various reasons, such as a disadvantageous energy compensation or the danger of a short-circuit of one or more energy stores due to simultaneous activation of two or more switches causing a direct closed circuit between the connections of the one or more said energy stores, or in the case of use in a sequence by creation of problematic transition states. According to this advantageous aspect of the invention, the codebook preferably contains only the states that are absolutely needed to provide the desired flexibility of the pulse form. So-called impermissible states, such as states in which two or more switches short-circuit an energy store given simultaneous or overlapping activation, are not included in the codebook and in principle therefore cannot be presented. If necessary, impermissible states can be specifically determined and removed from the codebook. The number of entries in the codebook yields the minimum data rate of the signals that the at least one control unit or at least one electronic circuit subordinate to this control unit must communicate to the modules. This signal communication can occur either purely parallel, purely serially, or mixed parallel serial according to the above statements. The codebook can be designed minimally, meaning that the minimal, usually binary word length is determined that is necessary to clearly present all entries of the codebook, i.e., all needed states. Without restriction of the generality, a word can be electronically transmitted purely parallel, purely serially, or mixed parallel serial according to the above statements. Alternatively, additional redundancy can be added to enable easy error detection or error correction. To enable easy implementation, parity codes and convolution codes in the sense of the invention are preferred.

If, for example in the case of a binary transmission, the number of entries in the codebook, and consequently the number of all needed states, does not correspond to the power of two, such redundancy occurs automatically.

The use of a codebook with a firmly defined maximum number of entries has the advantage that the maximum data rate to be expected of the encoded signal or signals is known, and in the sense of the invention is lower than the maximum data rate of the decoded switch control signals. Switch control signals describe the states of associated switches. As an example, for each associated switch they can comprise an on/off status bit.

In the sense of the invention, other source coding methods for reducing the average data rate and/or average redundancy of the coded signal can also be used [see J. Proakis (2001). Digital Communications. 4th edition, McGraw-Hill, Boston.]. Especially advantageous in the sense of the invention in particular are source coding methods that can ensure a maximum data rate.

Other advantages and embodiments of the invention emerge from the description and the attached drawings. The invention is shown schematically in the drawings based on embodiments, and is described with reference to the accompanying drawings schematically and thoroughly.

### BRIEF DESCRIPTION OF THE DRAWINGS

FIG. 1 shows a magnetic stimulation technology from the prior art that for the first time enables low flexibility of the pulse form.

FIG. 2 shows a further development of the magnetic stimulation technology from FIG. 1, in which it is possible to commutate between two high-voltage oscillators but no random pulses can be generated.

FIG. 3 shows a magnetic stimulation technology from the prior art that in principle can generate any pulse forms. For this N modules 301-304 are connected with a stimulation coil 305 such that the modules, through dynamic change of the interconnection of their energy stores with each other, including electrically in series and/or electrically in parallel, can generate a random voltage and/or current course at the outputs of the stimulation coil 305. The switches present in the modules are driven by at least one control unit through at least one electrical control bus 306.

FIG. 4 presents two exemplary modular circuits 401, 402 of the technology from FIG. 3. The modules contain at least one energy store 403, 404 and at least two semiconductor switches, in short switches 405-416, which can be implemented within a typical switch element, preferably field effect transistors are used. As a rule, the switches are supplemented by free-wheeling diodes and also with suppressor circuits (so-called snubbers). The switches 405, 406, 409-412 to the left of the energy store 403, 404 and their free-wheeling diodes and suppressor circuit are called side A without restriction of the generality. The switches 407, 408, 413-416 to the right of the energy store 403, 404 and their free-wheeling diodes and suppressor circuit are called side B without restriction of the generality.

FIG. 5 shows two exemplary adjacent modules according to the invention. The switches that can electrically connect the electrical energy stores 502, 503 of the two modules directly with each other together with their optional suppressor circuit 504 form the intermodule connection 501. Instead of the half-bridge arrangement of two switches that each module contributes to the intermodule connection, according to U.S. Ser. No. 13/990,463 each module of four switches in two half-bridges forms the share of the module at the inner module connection to enable a parallel circuit of energy stores of different modules. An intermodule connection 501, in turn, consists of at least two intermodule connection subunits, where the intermodule connection subunits each represent the interface of the intermodule connection with the participating modules. Consequently, the intermodule connection subunits with respect to a module in each case is the share of the corresponding module at the intermodule connection 501.

FIG. 6 shows five advantageous states of a particular embodiment in which the states are coded per intermodule connection. In the left column are shown the switches of the intermodule connection of two adjacent modules and their at least one energy store each, in the right column the equivalent electrical connections created by the switches and the corresponding states of the intermodule connection. The states shown in particular are serial-positive 601, serial-negative 602, bypass 603, passive 604, parallel 605 and a state 606 defined here as impermissible in which at least one energy store is short-circuited through two or more switches by corresponding activation of switches. The state mainly describes the form in which the system's electrical energy stores are electrically interconnected with each other. This form of interconnection is dynamically changeable in the sense of the invention.

FIG. 7 shows four typical states of a particular embodiment in which the states are coded per intermodule connection subunit. In the left column are shown the switches of the intermodule connection subunit of a module and its at least one energy store, in the right column the equivalent electrical connections created by the switches in the corresponding states of the intermodule connection unit. The states shown in particular are positive 701, negative 702, passive 703, parallel 704, and a state 705 usually impermissible in which at least one energy store is short-circuited through two or more switches by corresponding activation of switches.

FIG. 8 shows two exemplary codings of particular embodiments of the invention in which the states are coded per intermodule connection. With the aid of a suitable partially independent coding 801, three bits that are sufficient for the state of an intermodule connection with five different states can be divided such that two of the three bits, for example the first and the second bit, code the state of the one intermodule connection unit participating in the intermodule connection (see “to module 1”), while the third bit and one of the two named bits, for example the second and the third bit, together code the state of the other intermodule connection subunit participating in the intermodule connection (see “to module 2”). Consequently, the partially independent coding 801 is a skillful mixed solution between one coding per intermodule connection and one coding per intermodule connection subunit in the sense of the invention, in which each intermodule connection subunit must receive and decode only two bits instead of three bits that would be necessary for the clear definition of the intermodule connection with five different states in the codebook. For this, bits a and c can be arranged symmetrically as in FIG. 8 with the reference sign 801 so that all intermodule connection subunits can each use a decoder of the same type. Since the signals normally must be transmitted to the modules electrically isolated (for example, with optocouplers, capacitive, inductive or other galvanically separating signal transmitters), such a reduction of the amount of data, which leads to a reduction of the number of parallel lines and/or a reduction of the bit rate per line, is extremely advantageous. Alternatively, each third bit can also be evaluated by the respective decoder to serve as a check bit for detection of transmission errors.

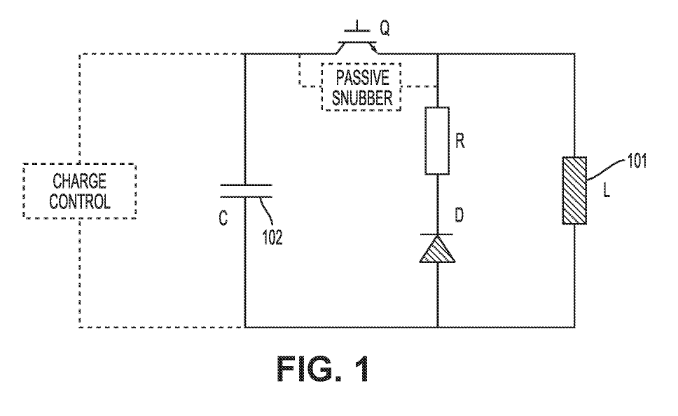

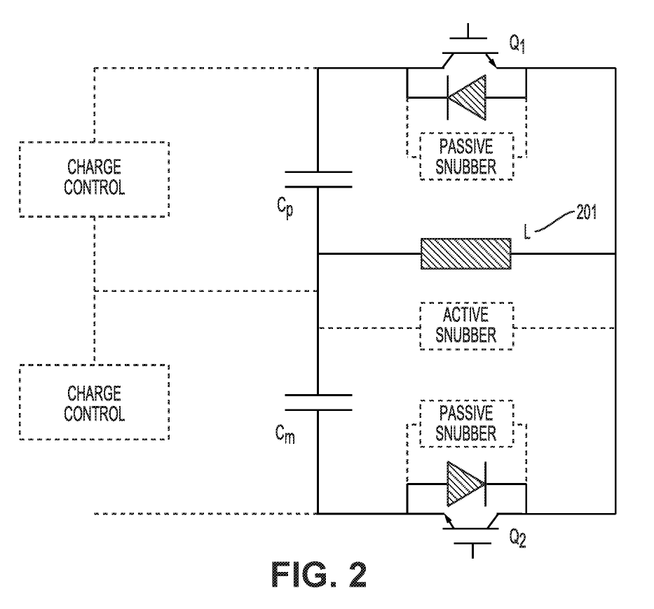

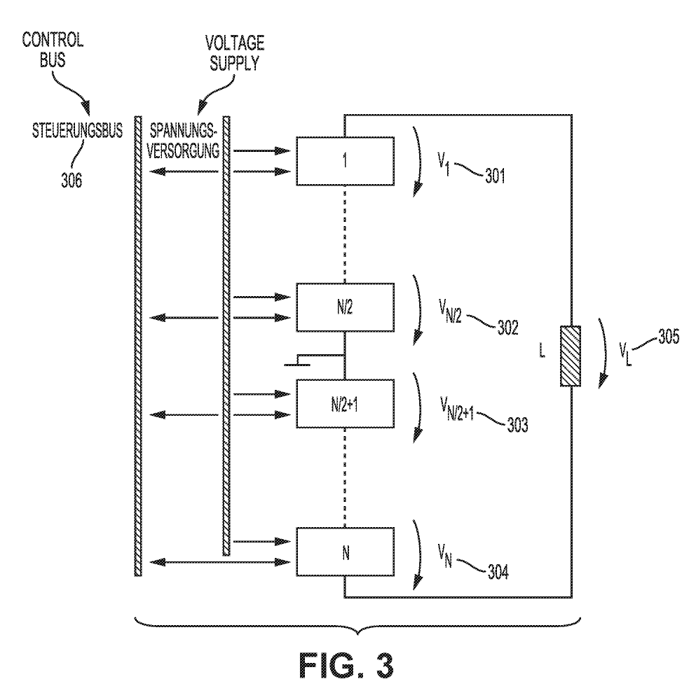

By contrast, if the “bypass” state for the intermodule connection is dispensed with, as in example 801, four states are present in the codebook per intermodule connection that need two binary bits for clear representation. While the code words for the clear description of the state of the intermodule connection are shorter than in the partially independent coding 801, each of the at least two intermodule connection subunits of the intermodule connection—if the decoding occurs through at least two independent decoders, at least one each for each intermodule connection subunit—needs both bits for the clear decoding and detection of which switches of the given intermodule connection subunit are to be activated. This thus yields the same number of bits per intermodule connection subunit as in the embodiment shown in Table 801. Such a decoding with at least two decoders can be very advantageous, since the decoding can be done before the electrical separation by galvanic transmitters before the decoding and thus a smaller amount of data must be transmitted galvanically separated.

The order of bits can be interchanged at will without restriction of the generality. The code can also be inverted, which means that 0 and 1 are interchanged.

FIG. 9 shows a particular embodiment of the invention that comprises at least one control unit 901, at least one galvanically separating signal transmitter 905, at least one decoder 907, and at least two modules 910, each comprising at least one electronic switch and at least one energy store. The modules are designed, for example, according to U.S. Pat. No. 7,269,037, DE 101 03 031, WO 2012/072197, DE 10 2010 052 934, WO 2012/072168, EP 2011/0179321, WO 2013/017186, DE 10 2011 108 920, DE 10 2016 112 250, and DE 2015 112 512, such that with the aid of at least one switch per module of the at least two modules named the electrical interconnection between at least two energy stores can switch dynamically at least between two of the following states: (a) electrical connection of the energy stores in series; (b) electrical connection of the energy stores parallel to each other; (c) bridging of at least one energy store so that no charge can flow in or out of the corresponding energy store.

This control unit sends electrical signals 902, for example through an electric bus, to at least one optional encoder 903 that encodes the signals such that the average amount of data and/or the average redundancy of the coded signals 904 is less than the corresponding average amount of data and/or the average redundancy of the uncoded or decoded switch control signals 909 and/or the average entropy of the coded signals 904 of the uncoded or decoded 909 is higher than that of the uncoded or decoded switch control signals 909. Under particular conditions, the maximum amount of data of the coded signals 904 is likewise less than the corresponding maximum amount of data of the uncoded or decoded switch control signals 909. The switch control signals 909 can be designed, for example, such that for each switch of a group of switches, for example assigned to decoder 907, at least one separate bit is provided that describes the state (electrically conductive closed vs. electrically non-conductive open) of the corresponding switch. The switch control signals 909 thus describe switch states of the individual switches.

As in all embodiments of the invention, the electrical signal connections and buses 902, 904, 906, 908, 909 without restriction can transmit serial, parallel, or mixed serial/parallel data.

At least one galvanically separating signal transmitter 905 isolates the electrical voltage level of the signals from the voltage level of the electrical control unit and/or other electronic components.

Optionally, between the at least one control unit 901 and the optional at least one encoder or coder 903 one of the at least one control units can contain a subordinate electronic circuit that receives electrical signals from the at least one control unit 901 or at least one last subordinate electronic circuit and itself in turn sends signals 902 to the at least one encoder or coder 903.

The order of the optional at least one encoder 903, the at least one galvanically separating signal transmitter 905, and the decoder 907 can be interchanged and/or partially integrated into the modules and/or divided into multiple parallel units, each processing either all or only a subset of all signals, for example only for one module each. Preferably, the decoding by the at least one decoder 907 always takes place after the optional coding by the optional at least one encoder 903.

The electrical power connection 911 between two modules 910 serves for the electrical energy transmission and electrical interconnection between the energy store elements of the associated modules, and in its design is usually adapted to the module type (e.g., M2C four-quadrant modules, see for example U.S. Pat. No. 7,269,037; M2C two-quadrant modules, see for example DE 101 03 031, M2SPC four-quadrant modules, see for example WO 2012/072197, DE 10 2010 052 934, DE 10 2011 108 920, WO 2013/017186; or M2SPC two-quadrant modules, see for example WO 2012/072168, US 2014/049230), and makes it possible with the aid of the at least one switch per module to change in their electrical interconnection among each other the at least one energy store of at least two modules 910. For example, the electrical power connection 911 for M2SPC modules is usually designed at least through two electrical connections to enable a parallel interconnection of modules 910 and electrical energy stores. Two modules 910 directly connected with each other electrically by an electrical power connection 911 are usually identified as neighbors.

FIG. 10 presents a particular embodiment of the invention in which the coded control signals 1004 are first decoded by at least one decoder 1007 before the decoded signals 1006 are isolated by at least one galvanically separating transmitter 1007 from the voltage level of at least the at least one control unit 1001.

FIG. 11 shows a particular embodiment of the invention in which at least one decoder 1108 is integrated into at least one module 1109.

FIG. 12 shows a particular embodiment that further comprises at least one channel coder 1212 and at least one channel decoder 1215. The at least one channel coder 1212 specifically adds to the signal redundancy with a particular code rate for the error detection and/or error correction [see J. Proakis (2001). Digital Communications. 4th edition, McGraw-Hill, Boston.]. The at least one channel decoder 1215 performs an error detection and/or error correction and extracts the signal. While the coding by at least one optional encoder 1203 reduces the redundancy and prevents impermissible states, e.g., as a rule short-circuits of an energy store, the channel decoding reduces the specifically added redundancy.

FIG. 13 shows as an example an embodiment that dispenses with the at least one optional encoder.

FIG. 14 shows a particular embodiment in which at least one galvanically separating transmitter 1405 per module, per intermodule connection, or per intermodule connection subunit is present, for example also integrated into the given module.

### DETAILED DESCRIPTION

A device for generating stimulation pulses for inductive neurostimulation according to a first embodiment of the invention comprises at least one stimulation coil and at least three similar modules, the connections of which are electrically connected with the stimulation coil and each able to assume multiple switching states.

In a particular embodiment of the invention, the passive state is presented in the code such that it corresponds to the state detected by the decoder or decoders if the at least one control unit or the at least one electronic circuit subordinate to it performing the coding is not operable. The at least one control unit or the at least one electronic circuit subordinate to it performing the coding is not operable if it is not supplied with the specified voltage, it is in the reset mode, or it has detected an error and caused an emergency shutdown.

In the case of a control bus with so-called pull-down resistance, this would be, for example, a permanent low signal on all present channels. In the case of a control bus with so-called pull-up resistance, this would be, for example, a permanent high signal on all present channels. The inventor realized that this is particularly advantageous. If an error shutdown occurs during a pulse, the bypass state of all or multiple modules represents the fastest way to discharge the magnetic field energy from the coil, since the rectifier diodes in the modules normally implemented reduce the current with the maximum possible voltage and transfer the energy to the energy store. At the same time, in the initialization of the system the modules before the at least one control unit or the at least one electronic circuit subordinate to it performing the coding should only be ready to deliver no energy from the system after the modules or their decoder or decoders.

Alternatively, in a particular embodiment the bypass state in which the voltage of 0 V is forced to the connections of the stimulation coil is presented in the code such that it corresponds to the state detected by the decoder or decoders if the at least one control unit or the at least one electronic circuit subordinate to it performing the coding is not operable. In the case of an error, any current still flowing is reduced due to the magnetic energy stored in the stimulation coil at the internal resistance of the coil and the other circuit. The inventor realized that this state has the advantage that this can minimize the voltage at the connections of the coil that in the event of an error might be damaged at the insulation and could therefore be touched by a user. Furthermore, the coil energy is converted to heat and is therefore no longer present in electrical form. For safety reasons, this can be advantageous relative to a storage in electrical stores and potentially defective modules or modules controlled by a defective control unit.

In a particular embodiment, at least some signals are not mapped binary, i.e., with two different electrical symbols, such as <high> and <low> or <positive> and <negative> or <low-ohm> and <high-ohm>; instead, higher-stage modulating methods with more than two symbols are used, such as multiple different voltage levels or other known transmission methods like phase shift keying, quadrature amplitude modulation or the like.

In a particular embodiment, the encoder and decoder are implemented in the at least one control unit or in at least one of each subordinate electronic circuit.

In a particular embodiment, the respective functions of the at least one encoder and/or at least one decoder are presented in the at least one control unit or in at least one electronic switch subordinate to each control unit, where the at least one control unit or the at least one electronic circuit subordinate to each control unit is a programmable control, and consequently is, for example, a microprocessor, a memory-programmable control, a signal processor, a field programmable gate array (FPGA), a complex programmable logic device (CPLD), a programmable array logic component (PGA), or a comparable circuit.

In a particularly preferred embodiment, compared to the particular embodiments named above the respective functions of both at least one encoder and also at least one decoder are presented in the at least one control unit or in at least a last subordinate electronic circuit, where the at least one control unit or the at least one last subordinate electronic circuit is a programmable control.

In a particular embodiment, the invention has no encoder even though coded signals are used for the control; consequently, they are signals that for example have a lower redundancy or a lower data length than the status signals of the set of switches, for example the gate signals with transistors. In this particular embodiment at least one control unit or at least one last subordinate electronic circuit generates directly coded signals that are converted by at least one decoder into control signals for at least one switch of the modules.

The inventor realized that a control can occur based on the coded states. An algebra can be developed with the states of modules, intermodule connections, intermodule connection subunits and other groupings of multiple switches that allows, for example, pulse width modulators and other switch modulators without bypass to generate directly coded signals through a dedicated encoder. The direct generation of coded signals in the sense of the invention, without the use of separate encoders, does not restrict the invention. An important feature of this aspect of the invention is not the coding but the use of at least one coded signal for controlling at least one switch of at least one module.

In a particular embodiment of the invention, at least one switch of at least one module is not controlled by a coded signal of at least one control unit or at least one last subordinate electronic circuit, but rather by an electronic signal reflecting the state of the switch—for example, a binary signal with a symbol representing the blocking state and a symbol representing the conductive state—of at least one control unit or at least one last subordinate electronic circuit, while at least one other switch of at least one module is driven by a signal coded in the sense of the invention of at least one control unit or at least one last subordinate electronic circuit.

In a particular embodiment of the invention, the redundancy of the coded control signals 904, 1004, 1104, 1204 is as high as or higher than the redundancy of the totality of the uncoded and decoded switch control signals 1209, 909; for example, in the form of the respective gate signal or a respective binary switch state description of the form to vs. from, of all individual switches of the modules together. This is especially in the sense of the invention here if the average redundancy of the coded control signals, based solely on a channel coding or an error detection/error correction code, for example parity bit or convolution code, is as high as or higher than said average redundancy of the totality of the uncoded or decoded switch control signals, for example the gate signals of the field effect transistors, IGBTs or the like, and the average redundancy of the coded control signals minus the channel code rate of the coded control signals [see J. Proakis (2001). Digital Communications. 4th edition, McGraw-Hill, Boston.] is lower than the average redundancy of the uncoded or decoded switch control signals 1209, 909.

In a particular embodiment of the invention, the coded signals have a known finite maximum data rate than is less than the maximum data rate of the uncoded or decoded switch control signals.

In an embodiment of the invention, the at least one decoder comprises at least one programmable logic component, for example a programmable array logic component (PGA), a complex programmable logic device (CPLD), a field programmable gate array (FPGA), or a comparable circuit that is able to implement at least one-channel electronic logic functions.

In another embodiment, at least one encoder comprises at least one programmable logic component.

In a particular embodiment, the said at least one programmable logic component is designed such that the state of at least one electronic input signal of the at least one programmable logic component at a particular time usually completely defines all electronic output signals of the at least one programmable logic component.

In a particular embodiment, said at least one programmable logic component is designed such that the state of at least one electronic output signal of the at least one programmable logic component is not completely defined by its electronic input signals at a particular time, but is fully defined by its electronic input signals at at least two particular times.

In a particular embodiment, said at least one programmable logic component is designed such that the state of at least one electronic output signal of at least one programmable logic component, besides the state of at least one input signal at at least one time, is also influenced by at least one state of at least one electronic output signal of the past.

In a particular embodiment, at least one of said possibly multiple programmable logic components is programmable exactly once, i.e., is changeable such that it permanently assumes the logic function necessary or advantageous for its operation that describes the connection of the at least one output signal and the at least one input signal.

In a particular embodiment, at least one of said possibly multiple programmable logic components is programmable multiple times. This embodiment is especially advantageous since the coding and communication with at least one control unit of devices in the field can be changed to, for example, provide them with a more advantageous encoding or decoding, expand their flexibility, change their control behavior, or change the codebook. In addition, in this way the components can be very easily adapted and used by programming for a second purpose (so-called second life) in another product or another product type, such as an energy engineering, medical technology, or automobile application.

In a particularly preferred embodiment, at least two of the modules, besides a serial state in which the electrical energy stores of two or more modules can be temporarily interconnected in series, necessarily have a parallel state in which the electrical energy stores of two or more modules can be temporarily electrically interconnected with each other in parallel.

### Coding on a Modular Basis

In a particular embodiment of the invention, the signals of at least three modules are coded independent of each other. This means the decoding of the encoded control signals, and thus the determination of which of the module's switches are to be activated, needs no information about the state of other modules. Consequently, the signals can be physically separated from other modules; for example, transmitted through independent parallel data lines assigned to the respective modules.

Preferably, each module has at least one dedicated data line assigned to it going from the at least one control unit or from the at least one electrical circuit subordinate to it performing the coding, through which the coded signals controlling the module are transmitted to said module.

In a particularly preferred embodiment, the codebook of the embodiment named above contains a maximum of four states {bypass, serial positive, serial negative, passive};

wherein in the bypass state at least one energy store of the module is not electrically connected with one of the adjacent modules;

wherein in the serial-positive state at least one energy store of the module with a previously determined polarity is electrically connected with the two adjacent modules such that the one contact of the energy store is electrically connected with an adjacent module and the other contact of the energy store is electrically connected with the other adjacent module;

wherein in the serial negative state the energy store of the module with polarity reversed relative to the serial positive state is electrically connected with the two adjacent modules such that the one contact of the energy store is electrically connected with an adjacent module and the other contact of the energy store is electrically connected with the other adjacent module;

wherein in the passive state the switches of the modules are either deactivated and only free-wheeling diodes conduct current, or alternatively, the switches are operated as rectifiers (so-called synchronous rectifiers).

In another particularly preferred embodiment, the codebook of the embodiment named above additionally contains the following states {parallel side A and serial positive side B, parallel side A and serial negative side B, parallel side A and parallel side B, parallel positive side A and parallel side B, serial negative side A and parallel side B};

wherein in the state <parallel side X and serial positive side Y> the switches of the side X of the module are activated such that at least one energy store of the module is electrically connected in parallel with the energy store of the module directly adjacent to side X and a previously defined contact of said energy store, for example the positive one, is electrically connected with the module directly adjacent to side Y; wherein in the state <parallel side X and serial negative side Y> the switches of the side X of the module are activated such that at least one energy store of the module is electrically connected with the energy store of the module directly adjacent to side X and the other contact of said energy store, for example the negative one, is electrically connected with the module directly adjacent to side Y;

wherein in the state <parallel side X and parallel side Y> the switches of the module are activated such that at least one energy store of the module is electrically connected with at least one energy store each of both adjacent modules and wherein X and Y can each be either A or B.

Another particularly preferred embodiment of the invention differs from the one named directly above in that the codebook necessarily contains no bypass state. As a result, the number of states corresponds to a power of two and can be transmitted very low-data on binary channels or data buses. The inventor realized that the bypass state is not necessary for creating the flexibility regarding the pulse form and its function can be mapped by other module states.

Another particularly preferred embodiment uses at least two bypass states, wherein a second bypass state, here identified as bypass inverse state without restriction of the generality, differs from a first bypass state by inversion of at least two electronic switches, by which in this second bypass state at least one energy store of the module is likewise not electrically connected with one of the adjacent modules even though the current flows through other electronic switches.

### Coding on an Intermodule Connection Basis

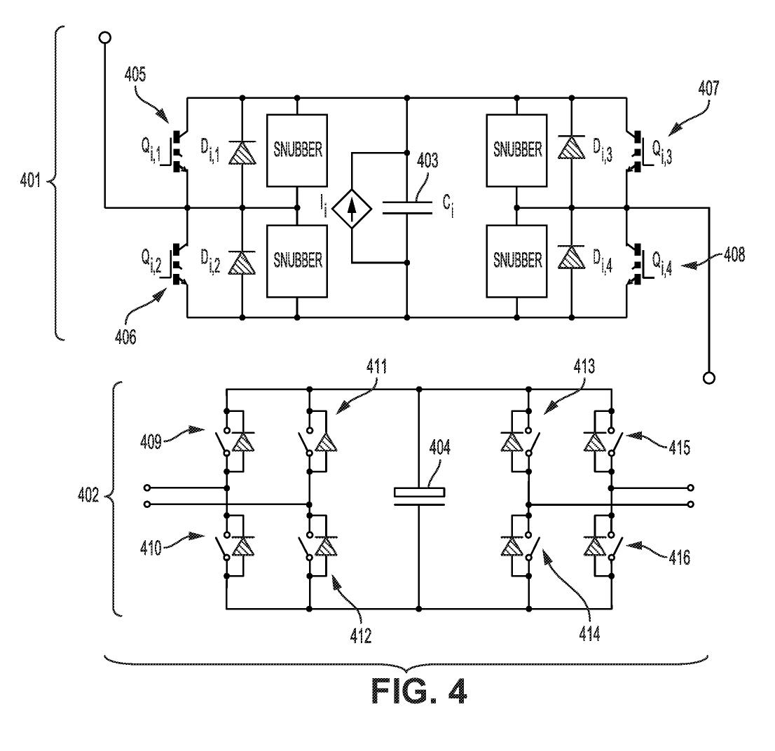

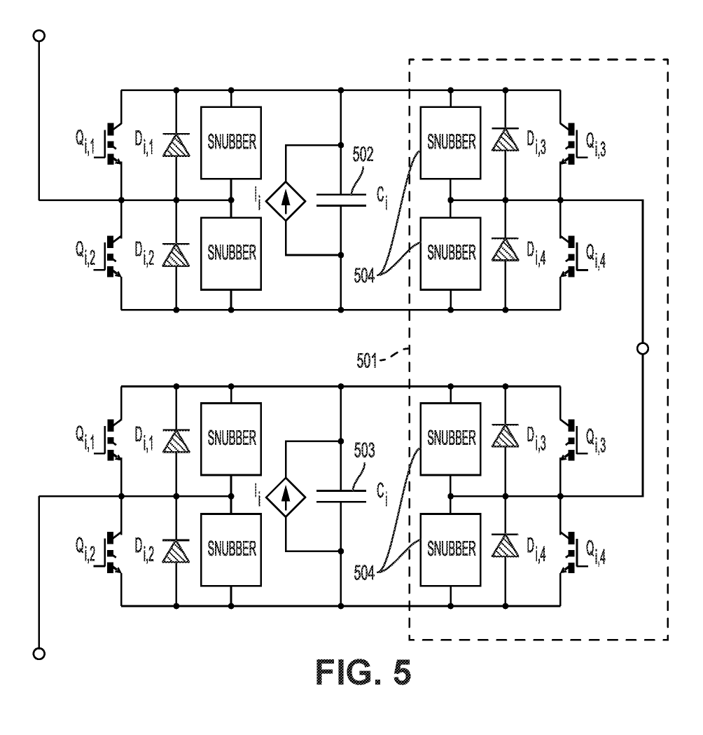

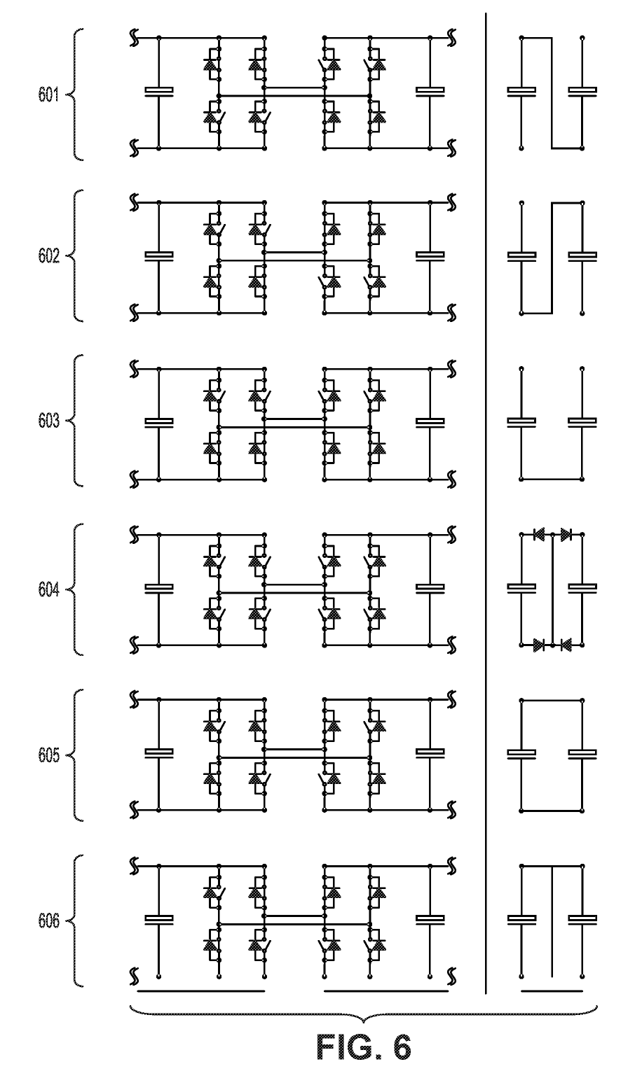

The coding per module described above is particularly well-suited if only serial, bypass, and passive states are used in the control but no parallel states. The reason for this can be that they are not needed for the application or are not implemented by the module type used (see for example the module type with reference sign 401 in FIG. 4). The inventor realized that with a use of parallel states differing codings are very advantageous. In the coding of states per module, module state combinations can be easily mapped in which one module can short-circuit the energy store of another. Such combinations have potential for data compression, and such combinations need not be contained in the code set of the codebook. One possibility that initially appears absurd because it unnaturally runs counter to module switch structures is the independent coding of intermodule connections, i.e., in each case the switches of two directly adjacent modules that enable the direct electrical connection of the energy stores of the two modules through suitable activation. Whereas at the module level up to nine important states should be considered in the codebook for flexible operation, analyses reveal that the number of states at the level of the intermodule connections can be reduced to five and fewer without restricting the flexibility of the overall system.

In a particular embodiment of the invention, the signals of each intermodule connection are coded independent of each other. This means the decoding of the coded control signals, and consequently the determination of which switches of the module are deactivated, needs no information about the state of other intermodule connections. An intermodule connection comprises only the switches of the two modules connected with each other through the intermodule connection that are necessary for presentation of all electrical connection states of the energy stores of the two said modules. Due to the independence from other intermodule connections, the control signals of the individual intermodule connections can be physically separated, for example transmitted through independent parallel data lines assigned to the respective intermodule connections.

Preferably, each intermodule connection receives at least one dedicated data line assigned to it from the at least one control unit or from the at least one electrical circuit subordinate to it performing the coding, through which the coded signals controlling the module are transmitted to said module. Particularly preferred, each intermodule connection receives at least two dedicated data lines belonging to it of which at least one data line furnishes signals to the part of the intermodule connection (so-called intermodule connection subunit) belonging to one of the two modules.

In a particularly preferred embodiment, the codebook of the embodiment named above contains for an intermodule connection a maximum of four states {bypass, serial positive, serial negative, passive};

wherein in the bypass state only one of the two electrical connections of at least one energy store of at least one of the modules connected by the intermodule connection is connected conductively with the equivalent electrical connection of the at least one energy store of at least one other of the modules connected with the intermodule connection, for example only the negative connections of the energy store are electrically connected while the positive ones remain electrically unconnected;

wherein in the serial positive state a previously determined electrical connection (for example, the positive one) of at least one energy store of one of the modules connected by the intermodule connection is connected electrically conducting with the non-equivalent (in the above example, consequently only the negative) electrical connection of at least one other of the modules connected by the intermodule connection;

wherein the serial negative state forms the inverse of the serial positive state and consequently a previously determined electrical connection (for example, the negative one) of at least one energy store of one of the modules connected by the intermodule connection is connected electrically conducting with the non-equivalent (in the above example, consequently, now the positive one) electrical connection of at least one other of the modules connected by the intermodule connection, and the electrically connected electrical connections of the connected energy stores do not correspond to those of the serial positive state;

wherein in the passive state the switches of the intermodule connection are either deactivated and only free-wheeling diodes conduct current or, alternatively, the switches are operated as rectifiers (so-called synchronous rectifiers).

In another particularly preferred embodiment, the codebook of the embodiment named above additionally contains at least one parallel state for the intermodule connection;

wherein the parallel state of the switch of the intermodule connection is activated such that at least one energy store of one of the modules connected by the intermodule connection is connected electrically parallel with at least one energy store of one other of the modules connected by the intermodule connection.

Another particularly preferred embodiment differs from the aforementioned embodiment in that the codebook contains no bypass state. The inventor realized that this can be replaced without substantial loss of flexibility of the overall system through other states, in particular the parallel state. This allows the number of states to be very easily brought to a power of two, so that the states with minimal redundancy can be coded in binary signals.

### Coding on an Intermodule Connection Subunit Basis

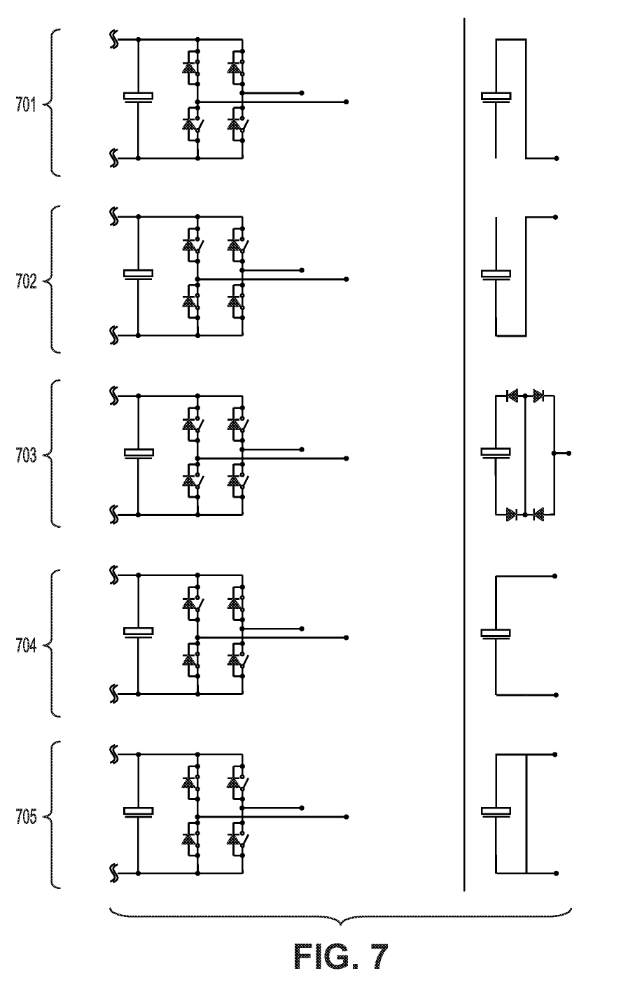

In a particular embodiment, each state of each intermodule connection subunit is coded separately. The codebook of these particular embodiments contains at least three states (positive, see FIG. 7 701, negative 702, passive 703);

wherein in the positive state one of the two electrical connections of at least one energy store of the module belonging to the intermodule connection subunit is electrically connected with at least one module connection of the intermodule connection subunit;

wherein in the negative state one of the two electrical connections of at least one energy store of the module belonging to the intermodule connection subunit is electrically connected with at least one module connection of the intermodule connection subunit;

wherein in the passive state the switches of the intermodule connection are either deactivated and only free-wheeling diodes conduct current, or alternatively the switches are operated as rectifiers (so-called synchronous rectifiers).

In a particularly preferred embodiment, the codebook compared to the aforementioned particular embodiment additionally contains a parallel state (704);

wherein in the parallel state each of the two electrical connections of at least one energy store of the associated module are electrically connected through corresponding activation of the switches of the intermodule connection subunit with another module connection of the intermodule connection unit.

Another particularly preferred embodiment differs from the aforementioned embodiment in that the codebook contains no bypass state. As already explained, the bypass state can be replaced without essential losses of flexibility of the entire system by other states, in particular the parallel state. This allows a clear reduction of the number of states so that the bit width of the signals can be reduced.

### Restriction of Multiple Separate Coding Units to a Partially Independent Coding

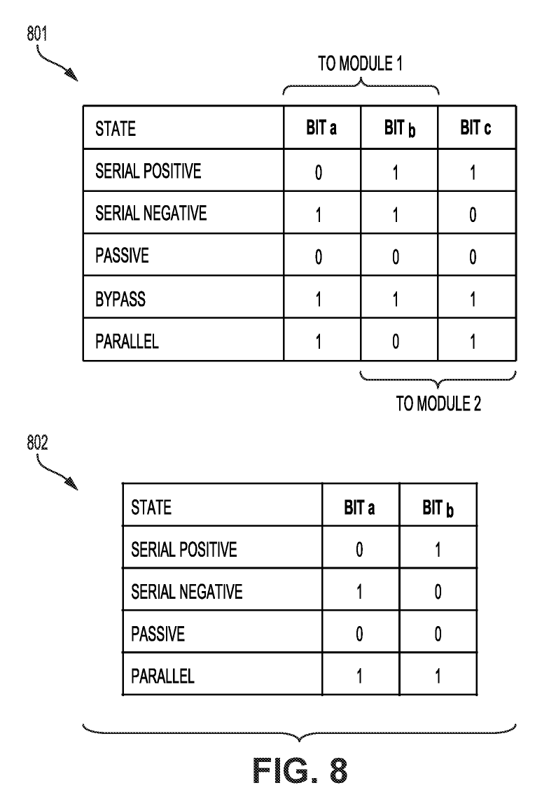

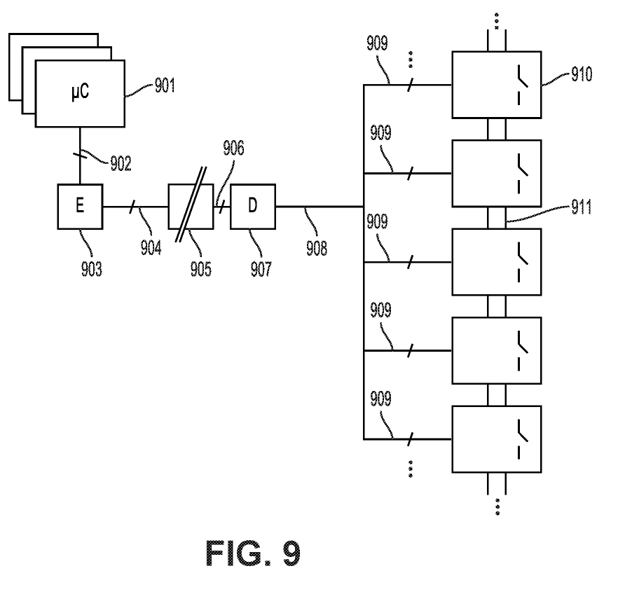

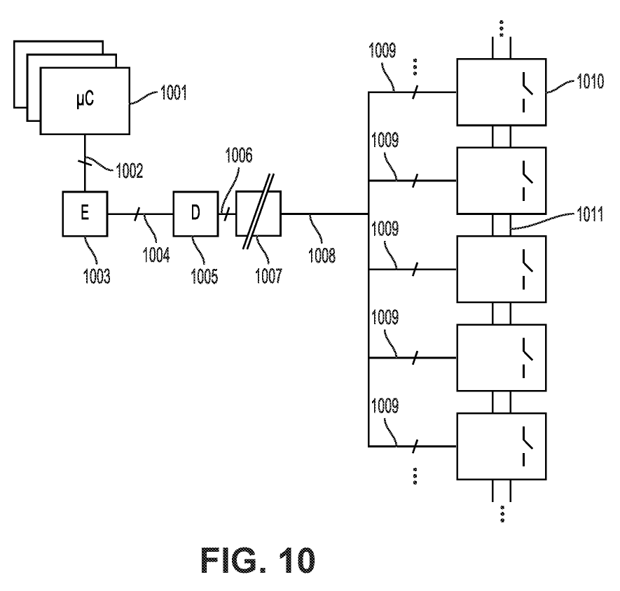

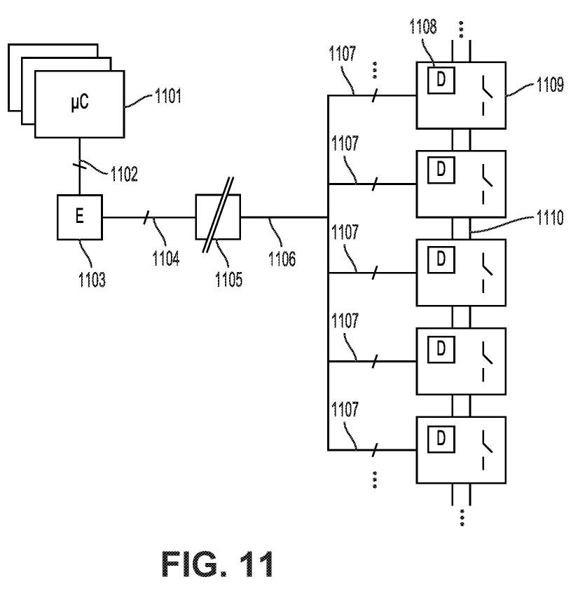

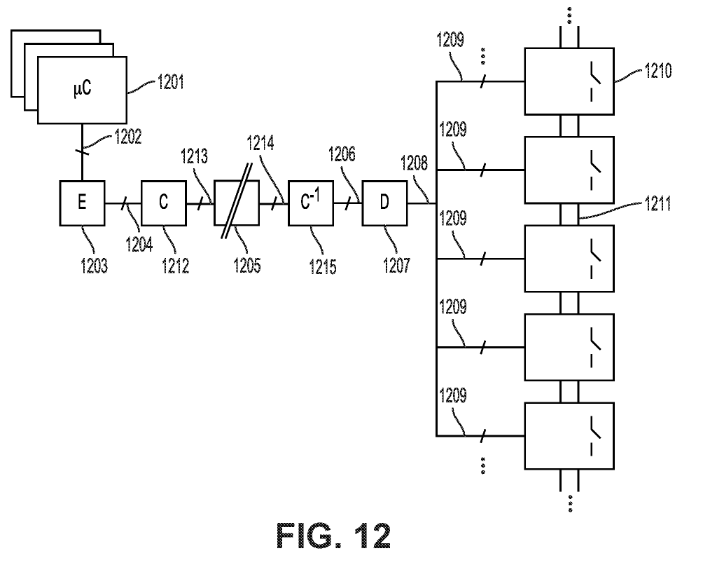

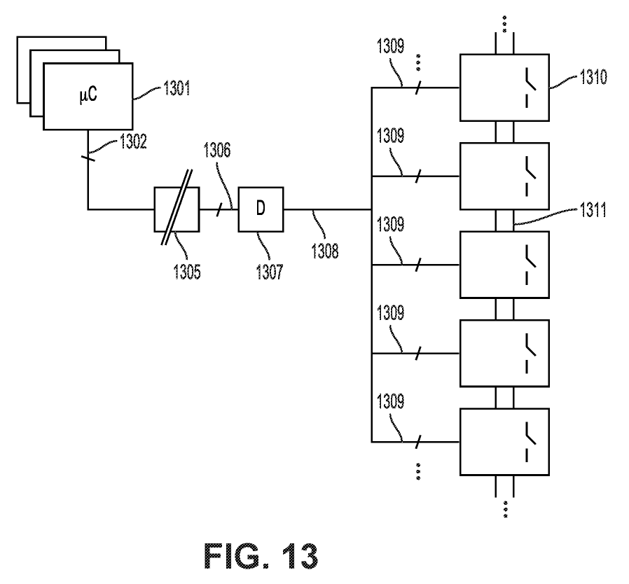

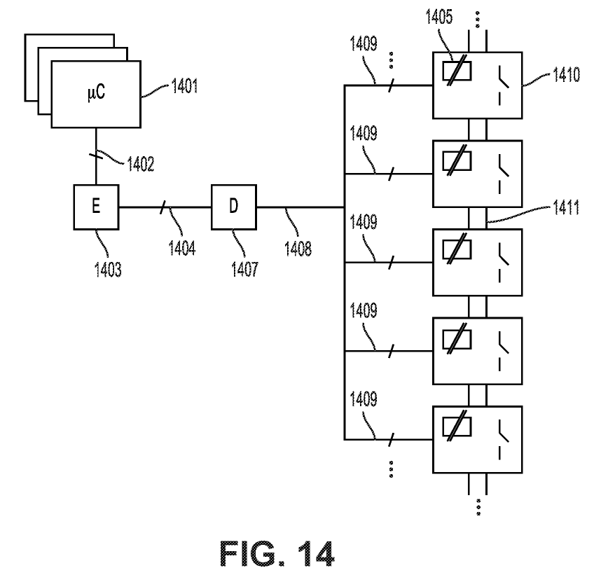

In an embodiment of the invention, the states of at least two different disjoint subunits of the system, such as modules, module groups, intermodule connections, and intermodule connection subunits, can be coded such that the coding of each of the subunits of the system is partially independent, meaning that at least a part of the shared signal of subunits disjoint for each of the at least two different subunits is necessary for clear determination of the respective state. As FIG. 8 shows, codes can be used, for example, that jointly code the states of the at least two different, disjoint subunits, for example an intermodule connection, such that for the clear determination of the respective state of each of these at least two different, disjoint subunits it is not the entire code word but only a part of it that is needed. However, as a rule a part of the code word of multiple of these at least two different, disjoint subunits is needed for the clear decoding of their state.

Relative to a coding separated for each intermodule connection subunit, this embodiment has the advantage that only a small number of signal channels must be galvanically separated. Signals can be galvanically separated through galvanically separating signal transmitters, also called isolating signal transmitters, such as optocouplers, capacitive signal transmitters, or comparable electrical components.

In addition, each subunit can use the signal of the at least one subunit with which its signals are partially independent for the error detection and/or error correction.

In a particular embodiment of the invention, the coding is performed such that at least two decoders receive as an input signal at least one signal the same for the at least two decoders as at least one so-called shared bit.

In a particularly preferred embodiment, this at least one shared bit is transmitted on a separate electronic signal line by the at least one control unit or at least an electronic circuit subordinate to this control unit. Preferably, the signal of this one separate electronic signal line can be generated with only one output pin of the at least one control unit or at least one electronic circuit subordinate to this control unit, and also transmitted in only one single signal line, and only be branched spatially close to the at least two decoders or looped through in the form of a bus. This allows technical resources to be conserved.

In a particularly preferred embodiment, this at least one shared bit determines the sign of the voltage, and consequently the polarity of each individual otherwise independent coded unit. This particularly preferred embodiment has the advantage that in many applications the modules, intermodule connections and the like can use the same polarity at any time without noteworthy losses of flexibility of the current generated and voltage courses.

In a particular embodiment, in each case the state of each half-bridge, which in each case consists of at least two series-connected electrical switches, is coded separately. The codebook of this particular embodiment contains at least three states (positive, negative, passive);

wherein in the positive state one of the two electrical connections of at least one energy store of the module belonging to the intermodule connection subunit is electrically connected with at least one module connection of the intermodule connection subunit;

wherein in the negative state one of the two electrical connections of at least one energy store of the module belonging to the intermodule connection subunit is electrically connected with at least one module connection of the intermodule connection subunit;

whereas in the passive state the switches of the intermodule connection are either deactivated and only free-wheeling diodes conduct current, or alternatively the switches are operated as rectifiers (so-called synchronous rectifiers).

The description of the preferred embodiments and the figures serve only as an exemplary explanation and illustration of the invention and the advantages achieved with it, but should not restrict the invention.

1. A method for generating short current pulses by means of an electronic circuit with at least two electrical switches and at least one electrical energy store, wherein at least one electronic control unit transmits electrical signals for controlling the at least two electrical switches that are coded on the basis of a predetermined pattern for switch states to be set of the at least two electrical switches, directed as coded electrical signals over an electrical signal transmission line to at least one decoder, and decoded by the at least one decoder into a respective switch state to be set of an individual one of the switch control signals describing at least two switches, wherein the respective switch control signals are directed to the respective at least two switches and there correspondingly converted, wherein at one output of the electrical switch for excitation of at least one stimulation coil current pulses with a total length of less than 5 ms are provided, so that the at least one stimulation coil generates magnetic field pulses with a magnetic flow density of 0.1 to 10 Tesla, which according to the principle of electromagnetic induction cause electrical currents in body tissue that through stimulation trigger at least an action potential of nerve and/or muscle cells, wherein the at least one stimulation coil is designed such that the magnetic field generated by it can penetrate the body tissue.

2. A method according to claim 1, wherein an average data rate or an average redundancy of the coded electrical signals is lower than a corresponding average data rate or an average redundancy of the signals decoded by the at least one decoder into switch control signals.

3. A device for generating short current pulses by means of an electronic circuit with at least two electrical switches and with at least one electrical energy store, whereas the device comprises at least: one electronic control unit configured to transmit electrical signals to control the at least two electrical switches, to be directed as coded electrical signals through an electrical signal transmission line to at least one decoder, the at least one decoder configured to decode the coded electrical signals into respective one switch state to be set of an individual one of the switch control signals describing two switches, whereas the respective switch control signals in the conversion at the respective at least two switches are such that at one output of the electronic circuit for stimulation of at least one stimulation coil current pulses with a total length of less than 5 ms are provided, so that the at least one stimulation coil given stimulation with these current pulses generates magnetic field pulses with a magnetic flow density of 0.1 to 10 Tesla, which according to the principle of electromagnetic induction cause electrical currents in body tissue that through stimulation trigger at least an action potential of nerve and/or muscle cells.

4. A device according to claim 3, wherein the average data rate or the average redundancy of the coded electrical signals is lower than the average corresponding data rate or the average redundancy of the signals decoded to switch control signals by the at least one decoder.

5. A device according to claim 3, comprising at least one coding unit configured to code electrical signals transmitted or to be transmitted by the electronic control unit based on a predetermined pattern for switch states to be set of the at least two electrical switches.

6. A device according to claim 5, wherein the at least one coding unit is integrated into the control unit or into at least one of at least one electronic circuit subordinate to an electronic control unit.

7. A device according to claim 5, wherein the at least one coding unit is a unit separate from the control unit and comprises at least one encoder.

8. A device according to claim 3, wherein the electronic circuit comprises at least two modules, which each comprise at least one electrical energy store and at least two electrical switches, wherein the at least two modules can assume at least two of the following switch states:
the at least one electrical energy store of a module is connected in series with the aid of the electrical switches with the at least one energy store of another module;
the at least one electrical energy store of a module is connected in parallel with the aid of the electrical switches with the at least one energy store of another module; and
the at least one electrical energy store of a module is circumvented with the aid of the electrical switches in the form of a bypass, which means that the at least one electrical energy store of a module is connected electrically conductive with at most only half of its at least two electrical contacts with an electrical energy store of another module and therefore no circuit with an electrical energy store of another module is present.

9. A device according to claim 8, wherein the device further comprises at least one galvanically separating signal transmission unit.

10. A device according to claim 9, wherein the galvanically separating signal transmission unit is configured to transmit at least a part of the coded electrical signals.

11. A device according to claim 8, wherein the device comprises at least one decoder per module, wherein a respective decoder of a module is designed to receive in each case only a subset of the totality of the coded electrical signals received from the decoders, and/or wherein the device comprises at least one decoder per intermodule connection, wherein a respective decoder of an intermodule connection is designed to receive in each case only a subset of the totality of the coded electrical signals received from decoders, and/or wherein the device comprises at least one decoder per intermodule connection unit, wherein a respective decoder of an intermodule connection unit is designed to receive in each case only a subset of the totality of the coded electrical signals received from decoders.

12. A device according to claim 11, wherein the respective subsets of coded electrical signals received from different decoders are not identical; in particular, they are pairwise disjoint.

13. A device according to claim 3, wherein the device further comprises at least one channel coder.

14. A device according to claim 13, wherein the at least one channel coder is integrated into the at least one electronic control unit or into at least one of the electronic circuits subordinate to at least one electronic control unit.
